(19) United States
(12) Patent Application Publication
(10) Pub. No.: US 2025/0264411 A1
(43) Pub. Date: Aug. 21, 2025

(54) METHODS AND COMPOSITIONS FOR PARTITIONING A BIOLOGICAL SAMPLE

(71) Applicant: 10x Genomics, Inc., Pleasanton, CA (US)

(72) Inventor: Felice Alessio Bava, Rome (IT)

(21) Appl. No.: 19/179,536

(22) Filed: Apr. 15, 2025

Related U.S. Application Data
(63) Continuation of application No. 18/450,746, filed on Aug. 16, 2023.
Application No. 18/450,746 is a division of application No. 17/172,709, filed on Feb. 10, 2021, now Pat. No. 11,835,462.
(60) Provisional application No. 62/975,168, filed on Feb. 11, 2020.

(51) Int. Cl. G01N 21/64; C12Q 1/6837

(52) U.S. Cl. CPC G01N 21/6458; C12Q 1/6837; C12Q 2563/107

(57) ABSTRACT

This disclosure relates to compositions and methods for analyzing a tissue section from a biological sample.

### CROSS-REFERENCE TO RELATED APPLICATION

This application is a continuation application of U.S. Patent Application of Ser. No. 18/450,746, filed on Aug. 16, 2023, which is a divisional application of U.S. Patent Application of Ser. No. 17/172,709, now U.S. Pat. No. 11,835,462, filed Feb. 10, 2021, which claims priority to U.S. Provisional Patent Application No. 62/975,168, filed Feb. 11, 2020. This application is incorporated herein by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Tissue sections have previously been studied by placing them on a slide during early steps of sample processing, where subsequent steps have to be performed on the slide, thus limiting potential future manipulations for tissue processing.

### SUMMARY

Disclosed herein are methods and compositions for manipulating a biological sample and determining the presence of an analyte in the biological sample. In some instances, provided herein is a method of determining abundance and location of an analyte in a biological sample, the method comprising: (a) embedding a plurality of sections of the biological sample into a polymer solution; (b) generating a plurality of partitions, wherein a partition of the plurality of partitions comprises a section of the plurality of sections of the biological sample; (c) manipulating each section in order to detect the analyte; and (d) determining the abundance and location of the analyte in a biological sample.

In some embodiments, provided herein are methods of processing a biological sample, the method including: (a) depositing a plurality of tissue sections obtained from the biological sample into a polymer solution; (b) generating a plurality of partitions wherein a partition of the plurality of partitions includes a tissue section of the plurality of tissue sections; and (c) imaging the tissue section of the plurality of tissue sections. In some embodiments, the tissue section is obtained from a fresh tissue sample or a frozen tissue sample. In some embodiments, the tissue section is obtained from a tissue sample that has been fixed. In some embodiments, the tissue sample is fixed in one or more of: paraffin, a wax, a resin, an epoxy, an agar, a glycol, a hydrogel, or a combination thereof. In some embodiments, the plurality of tissue sections are serial sections from a tissue sample. In some embodiments, the average thickness of the plurality of tissue sections is about 0.1 to about 100 micrometers. In some embodiments, methods of processing a biological sample provided herein further include fixing the tissue section prior to step (a). In some embodiments, the tissue section is obtained from a tissue sample that has been permeabilized. In some embodiments, methods of processing a biological sample provided herein further include permeabilizing the tissue section prior to step (a). In some embodiments, methods of processing a biological sample provided herein further include fixing the tissue section prior to permeabilizing. In some embodiments, methods of processing a biological sample provided herein further include permeabilizing the tissue section prior to or after step (b). In some embodiments, the tissue section is permeabilized via electroporation. In some embodiments, the tissue section is permeabilized via by contacting the tissue section with a permeabilization agent.

In some embodiments, the polymer solution is a hydrogel solution. In some embodiments, methods of processing a biological sample provided herein includes forming a hydrogel matrix from the hydrogel solution, and wherein generating a plurality of partitions includes generating a plurality of hydrogel macrobeads, wherein a hydrogel macrobead of the plurality of hydrogel macrobeads includes the tissue section. In some embodiments, the hydrogel macrobeads are generated by dissociating the hydrogel matrix. In some embodiments, methods of processing a biological sample provided herein further include removing the hydrogel matrix prior to imaging.

In some embodiments of methods of processing a biological sample provided herein, the capture probe binds to a biological analyte in the tissue section. In some embodiments, the partition of the plurality of partitions further includes an analyte capture agent. In some embodiments, the analyte capture agent binds to a biological analyte in the tissue section. In some embodiments, methods of processing a biological sample provided herein further include staining the tissue section in the partition. In some embodiments, the staining includes labeling two or more biological analytes in the tissue section with an optical label. In some embodiments, the two or more biological analytes are, individually, RNA, DNA, or protein. In some embodiments, the optical label is a fluorescent, radioactive, chemiluminescent, calorimetric, or colorimetric detectable label. In some embodiments, the staining includes immunohistochemical staining or chemical staining. In some embodiments, the partition is placed in a staining solution. In some embodiments, methods of processing a biological sample provided herein further include depositing the partition on a substrate. In some embodiments, the substrate is a multi-well plate. In some embodiments, the substrate is positively charged. In some embodiments, the partition is attached to the substrate via a chemical linker. In some embodiments, methods of processing a biological sample provided herein further include applying heat to the substrate. In some embodiments, the heat is applied to a portion of the substrate corresponding to the location of the partition. In some embodiments, the tissue section is imaged using bright field microscopy, fluorescence microscopy, or capillary microscopy.

In some embodiments, the methods further include dispensing the section onto a surface and imaging the biological sample. In some embodiments, the imaging comprises capillary microscopy, brightfield microscopy, or fluorescent microscopy.

In some embodiments, the polymer solution comprises a hydrogel.

In some embodiments, the biological sample is a tissue section sample. In some embodiments, the biological sample is from a fresh tissue sample, a frozen tissue sample, or a formalin-fixed, paraffin embedded (FFPE) sample. In some embodiments, the plurality of sections are serial sections from the biological sample. In some embodiments, the average thickness of the plurality of sections is about 0.1 to about 100 micrometers.

In some embodiments, the methods further include fixing the biological sample. In some embodiments, the methods further include permeabilizing the biological sample.

Also disclosed herein are kits. In some instances, the kit includes (a) a polymer solution comprising a hydrogel; (b) a container for the polymer solution; (c) one or more non-aqueous droplets to partition a biological sample comprising an analyte; (d) one or more compositions to manipulate the biological sample, wherein the one or more compositions are selected from the group consisting of: (1) a plurality of capture probes, wherein a capture probe of the plurality comprises (i) a spatial barcode and (ii) a capture domain that binds to a sequence present in the analyte; (2) a plurality of analyte capture agents, wherein an analyte capture agent of the plurality of analyte capture agents comprises: an analyte binding moiety that binds to the analyte; an analyte binding moiety barcode that uniquely identifies an interaction between the analyte and the analyte binding moiety; and an analyte capture sequence, wherein the analyte capture sequence binds to a capture domain; and (3) a protein-binding molecule for immunofluorescence or immunohistochemistry; and (e) instructions for performing any of the methods disclosed herein. In some instances, the analyte is an mRNA molecule or a protein.

Also disclosed herein are compositions. In some instances, the compositions include (a) a biological sample embedded in a polymer solution; (b) a non-aqueous droplet, wherein the non-aqueous droplet surrounds the biological sample; (c) one or more reagents to manipulate the biological sample, wherein the one or more reagents are selected from the group consisting of: (1) a plurality of capture probes, wherein a capture probe of the plurality comprises (i) a spatial barcode and (ii) a capture domain that binds to a sequence present in the analyte; (2) a plurality of analyte capture agents, wherein an analyte capture agent of the plurality of analyte capture agents comprises: an analyte binding moiety that binds to the analyte; an analyte binding moiety barcode that uniquely identifies an interaction between the analyte and the analyte binding moiety; and an analyte capture sequence, wherein the analyte capture sequence binds to a capture domain; and (3) a protein-binding molecule for immunofluorescence or immunohistochemistry; and (d) a substrate comprising a plurality of probes that is capable of detecting the analyte, the capture probe, or the analyte capture agent.

In some embodiments of methods of processing a biological sample provided herein, generating a plurality of partitions includes generating a plurality of droplets including the polymer solution, wherein a droplet of the plurality of droplets includes the tissue section. In some instances, the generating a plurality of partitions comprises surrounding the section with a non-aqueous droplet. In some embodiments, the plurality of droplets are formed in an emulsion including a non-aqueous droplet solution. In some embodiments, the non-aqueous droplet solution includes oil. In some embodiments, the plurality of droplets are generated in a microfluidic device. In some embodiments, the average volume of the plurality of droplets is less than 10,000 picoliters. In some embodiments, the average volume of the plurality of droplets is less than 1,000 picoliters. In some embodiments, the partition of the plurality of partitions further includes a bead including a capture probe.

In some embodiments, the analyte is an mRNA molecule. In some embodiments, the manipulating step comprises: (a) contacting the biological sample with a plurality of capture probes, wherein a capture probe of the plurality comprises (i) a spatial barcode and (ii) a capture domain that binds to a sequence present in the analyte; (b) hybridizing the analyte to the capture domain; (c) extending a 3′ end of the capture probe using the analyte that is bound to the capture domain as a template to generate an extended capture probe; and (d) amplifying the extended capture probe. In some embodiments, the determining comprises determining (i) all or a portion of the sequence of the spatial barcode or the complement thereof, and (ii) all or a portion of the sequence of the analyte; and using the determined sequences of (i) and (ii) to identify the location of the analyte in the biological sample, thereby determining the abundance and the location of the analyte.

In some embodiments, the analyte is a protein. In some embodiments, the manipulating step comprises attaching the biological sample with a plurality of analyte capture agents, wherein an analyte capture agent of the plurality of analyte capture agents comprises: (i) an analyte binding moiety that binds to the analyte; (ii) an analyte binding moiety barcode that uniquely identifies an interaction between the analyte and the analyte binding moiety; and (iii) an analyte capture sequence, wherein the analyte capture sequence binds to a capture domain. In some embodiments, the determining step comprises determining the abundance and location of the analyte, the method comprising: (a) contacting the biological sample with a substrate, wherein the substrate comprises a plurality of capture probes, wherein a capture probe of the plurality of capture probes comprises (i) the capture domain and (ii) a spatial barcode; (b) hybridizing the analyte to the capture probe; and (c) determining (i) all or a part of a sequence corresponding to the analyte binding moiety barcode, and (ii) all or a part of a sequence corresponding to the spatial barcode, or a complement thereof, and using the determined sequence of (i) and (ii) to identify the abundance and spatial location of the analyte in the biological sample.

In some embodiments, the manipulating comprises immunofluorescence or immunohistochemistry.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, wherein the cleaved capture probe can enter into a non-permeabilized cell and bind to target analytes within the sample.

FIG. 3 is a schematic diagram of an exemplary multiplexed spatially-barcoded feature.

FIG. 4 is a schematic diagram of an exemplary analyte capture agent.

FIG. 5 is a schematic diagram depicting an exemplary interaction between a feature-immobilized capture probe 524 and an analyte capture agent 526.

FIGS. 6A-6C are schematics illustrating how streptavidin cell tags can be utilized in an array-based system to produce a spatially-barcoded cells or cellular contents.

FIG. 7 shows an example of a microfluidic channel structure 700 for partitioning dissociated sample (e.g., biological particles or individual cells from a sample).

FIG. 8A shows an example of a microfluidic channel structure 800 for delivering spatial barcode carrying beads to droplets.

FIG. 8B shows a cross-section view of another example of a microfluidic channel structure 850 with a geometric feature for controlled partitioning.

FIG. 8C shows an example of a workflow schematic.

FIG. 9 is a workflow schematic illustrating exemplary steps of generating tissue macrobeads or droplets for various types of imaging analysis.

### DETAILED DESCRIPTION

### I. Introduction

Spatial analysis methods using capture probes and/or analyte capture agents provide information regarding the abundance and location of an analyte (e.g., a nucleic acid or protein). The efficiency of spatial analysis using arrays with capture probes depends, at least in part, on the density of the probes on the array or the density of the analytes captured on the array. That is, on how many capture probes can be printed on the surface of a slide or how many RNA molecules can be captured. Disclosed herein are methods and compositions for increasing the efficiency of spatial analysis by increasing the number of interactions between the capture probe and the analyte. In this way, analyte detection signal is increased, thus increasing the capturing efficiency, sensitivity, and the resolution of detection on the spatial array.

Traditionally, these methods identify a singular molecule at a location. Extending these methods to study interactions between two or more analytes would provide information on the interactions between two or more analytes at a location in a biological sample. Analyte capture agents as provided herein comprise an analyte binding moiety affixed to an oligonucleotide. The oligonucleotide comprises a sequence that uniquely identifies the analyte and moiety. Further, nearby oligonucleotides affixed to a different moiety in a nearby location can be ligated to the first oligonucleotide and then can be detected using the spatial methods described herein. The methods disclosed herein thus provide the ability to study the interaction between two or more analytes in a biological sample.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

In some instance, the capture domain is designed to detect one or more specific analytes of interest. For example, a capture domain can be designed so that it comprises a sequence that is complementary or substantially complementary to one analyte of interest. Thus, the presence of a single analyte can be detected. Alternatively, the capture domain can be designed so that it comprises a sequence that is complementary or substantially complementary to a conserved region of multiple related analytes. In some instances, the multiple related analytes are analytes that function in the same or similar cellular pathways or that have conserved homology and/or function. The design of the capture probe can be determined based on the intent of the user and can be any sequence that can be used to detect an analyte of interest. In some embodiments, the capture domain sequence can therefore be random, semi-random, defined or combinations thereof, depending on the target analyte(s) of interest.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434): 1463-1467, 2019; Lee et al., Nat. Protoc. 10(3): 442-458, 2015; Trejo et al., PLOS ONE 14(2): e0212031, 2019; Chen et al., Science 348(6233): aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

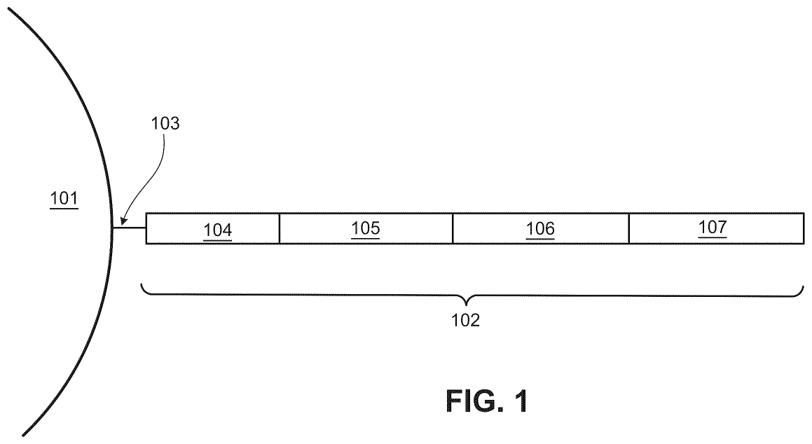

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

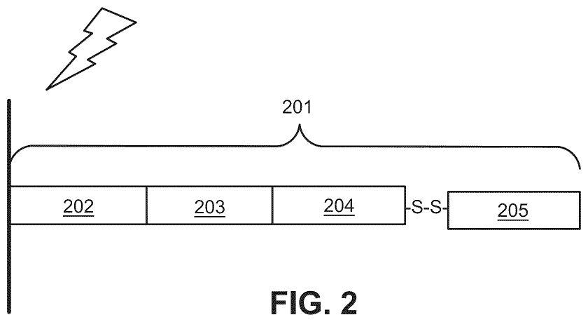

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

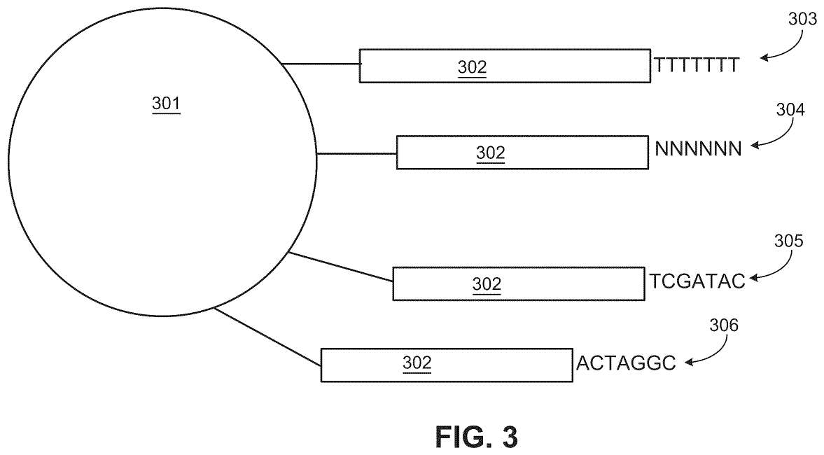

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

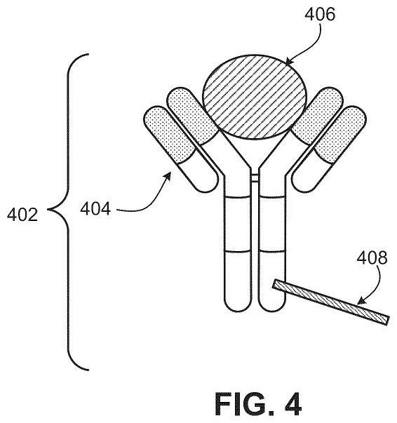

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. The analyte-binding moiety barcode domain 408 can comprise an analyte binding moiety barcode and a capture handle sequence described herein. The analyte-binding moiety 404 can include a polypeptide and/or an aptamer. The analyte-binding moiety 404 can include an antibody or antibody fragment (e.g., an antigen-binding fragment).

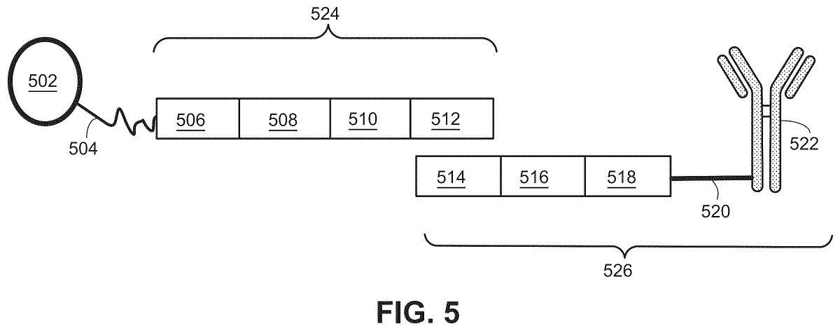

FIG. 5 is a schematic diagram depicting an exemplary interaction between a feature-immobilized capture probe 524 and an analyte capture agent 526. The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the capture agent barcode domain 516 to couple to the analyte binding moiety 522.

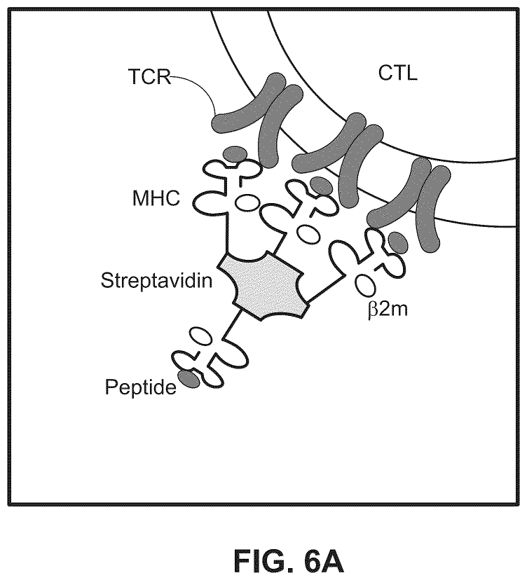

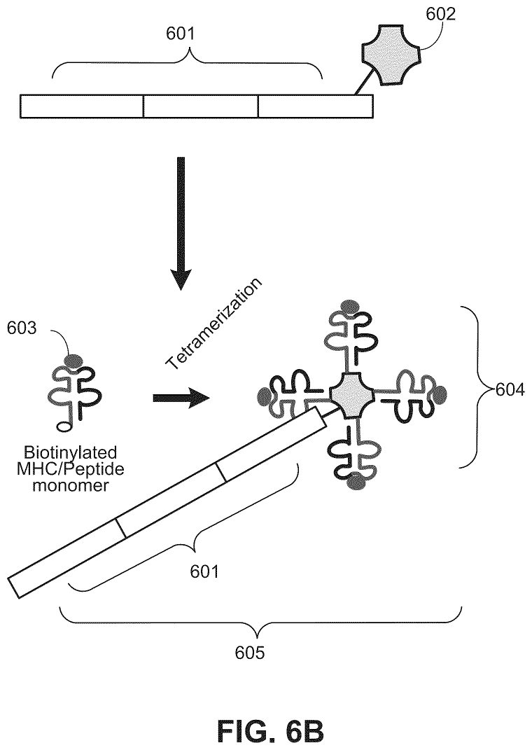

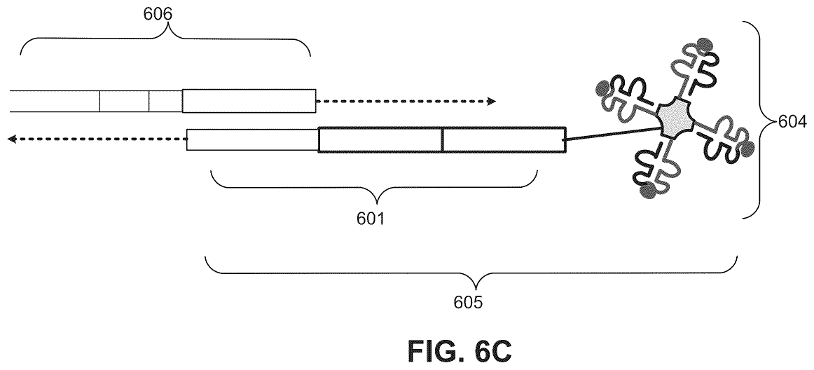

FIGS. 6A, 6B, and 6C are schematics illustrating how streptavidin cell tags can be utilized in an array-based system to produce a spatially-barcoded cell or cellular contents. For example, as shown in FIG. 6A, peptide-bound major histocompatibility complex (MHC) can be individually associated with biotin (β2m) and bound to a streptavidin moiety such that the streptavidin moiety comprises multiple pMHC moieties. Each of these moieties can bind to a TCR such that the streptavidin binds to a target T-cell via multiple MCH/TCR binding interactions. Multiple interactions synergize and can substantially improve binding affinity. Such improved affinity can improve labelling of T-cells and also reduce the likelihood that labels will dissociate from T-cell surfaces. As shown in FIG. 6B, a capture agent barcode domain 601 can be modified with streptavidin 602 and contacted with multiple molecules of biotinylated MHC 603 such that the biotinylated MHC 603 molecules are coupled with the streptavidin conjugated capture agent barcode domain 601. The result is a barcoded MHC multimer complex 605. As shown in FIG. 6B, the capture agent barcode domain sequence 601 can identify the MHC as its associated label and also includes optional functional sequences such as sequences for hybridization with other oligonucleotides. As shown in FIG. 6C, one example oligonucleotide is capture probe 606 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 606 may at first be associated with a feature (e.g., a gel bead) and released from the feature. In other embodiments, capture probe 606 can hybridize with a capture agent barcode domain 601 of the MHC-oligonucleotide complex 605. The hybridized oligonucleotides (Spacer C C C and Spacer rGrGrG) can then be extended in primer extension reactions such that constructs comprising sequences that correspond to each of the two spatial barcode sequences (the spatial barcode associated with the capture probe, and the barcode associated with the MHC-oligonucleotide complex) are generated. In some cases, one or both of these corresponding sequences may be a complement of the original sequence in capture probe 606 or capture agent barcode domain 601. In other embodiments, the capture probe and the capture agent barcode domain are ligated together. The resulting constructs can be optionally further processed (e.g., to add any additional sequences and/or for clean-up) and subjected to sequencing. As described elsewhere herein, a sequence derived from the capture probe 606 spatial barcode sequence may be used to identify a feature and the sequence derived from spatial barcode sequence on the capture agent barcode domain 601 may be used to identify the particular peptide MHC complex 604 bound on the surface of the cell (e.g., when using MHC-peptide libraries for screening immune cells or immune cell populations).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligation products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up-and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21;45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly (A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease (e.g., RNAse H). The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

The sandwich process is described in PCT Patent Application Publication No. WO 2020/123320, which is incorporated by reference in its entirety.

### II. Methods and Compositions for Spatial Analysis in Solution

Disclosed herein are methods and compositions for performing spatial analysis and analyte detection in a biological sample. In particular, the methods and compositions disclosed herein relate to manipulation of biological samples that can be performed while the biological sample is not on a surface, such as a glass slide. Generally, spatial analysis of analytes in biological samples (e.g., tissues) is coupled to microscopy readouts; because microscopy readouts require biological samples to be placed onto a surface (e.g., a slide), the biological sample usually is placed on the surface. Further, because scientists usually apply a biological sample on a surface (e.g., a glass slide) in the early steps of sample processing, so that all follow-up steps are also performed on the surface. For example, follow-up steps include analyte detection and sample manipulation, such as fluorescent tagging of proteins. The methods and compositions disclosed herein provide a way to detect analytes and manipulate the biological sample without the need to perform such steps on a surface. Further, the methods and compositions provided herein allow for high-throughput analysis of multiple sections of the same sample (e.g., using multiple tissue slices).

Accordingly, provided herein are methods of analyzing a biological sample, including examining the abundance and location of one or more analytes in the biological sample. In some instances, provided herein are various methods of processing a biological sample, the methods including: (a) depositing a plurality of tissue sections obtained from the biological sample into a polymer solution; (b) generating a plurality of partitions wherein a partition of the plurality of partitions comprises a tissue section of the plurality of tissue sections; and (c) imaging the tissue section of the plurality of tissue sections.

In some instances, the methods disclosed herein include methods of determining the abundance and the location of an analyte in a biological sample. In some instances, the methods include (a) embedding a plurality of sections of the biological sample into a polymer solution; (b) generating a plurality of partitions, wherein a partition of the plurality of partitions comprises a section of the plurality of sections of the biological sample; (c) manipulating each section in order to detect the analyte; and (d) determining the abundance and location of an analyte in a biological sample. Also disclosed herein are kits and compositions relating to the same.

Additional embodiments of the disclosure are provided herein.

### A. Biological Sample, Analytes, and Preparation of the Same

### (i) Biological Samples and Analytes

As used herein, a “biological sample” (also called “sample,” and where appropriate, “tissue sample,” “cell culture sample,” and the like) is obtained from the subject for analysis using any of a variety of techniques including, but not limited to, biopsy, surgery, and laser capture microscopy (LCM), and generally includes cells and/or other biological material from the subject. In some instances, a biological sample can be obtained from a eukaryote. In some instances, the biological sample is obtained from a mammal. In some instances, the biological sample is obtained from a human.

The biological samples described herein can be obtained from a tissue sample processed using any of the methods provided herein. The biological sample can be, e.g., a fresh tissue sample or a tissue sample that has been fixed. Fixed biological samples can be embedded in any suitable medium described herein or known in the art, including but not limited to, paraffin, waxes, resins, epoxies, agar, glycols, hydrogel or combinations thereof. After sectioning the biological sample into one or more tissue sections, the medium can optionally be removed from the tissue sections (e.g. deparaffinized) prior to depositing them into the polymer solution. The tissue sections can also be obtained from a frozen biological sample (e.g. snap frozen in liquid nitrogen), by methods provided herein or known in the art (e.g. sectioned on a microtome or cryostat). Frozen tissue sections can be thawed (completely or partially) prior to depositing into the polymer solution. The biological sample may be (or can have been) permeabilized prior to obtaining tissue sections, using methods provided herein or known in the art (e.g. using any of the variety of permeabilization agents and/or conditions, e.g., electroporation, described herein or known in the art).

Biological sections can also be fixed and/or permeabilized prior to being deposited into the polymer solution, using methods provided herein or known in the art. In some instances, the tissue sections are fixed prior to or after permeabilization. In some instances, tissue sections are permeabilized after being deposited into the polymer solution, and before or after the generation of partitions in step (b) of the methods provided herein.

In some embodiments, a biological sample can be permeabilized to facilitate transfer of analytes out of the sample, and/or to facilitate transfer of species (such as capture probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution). In some embodiments, the biological sample can be permeabilized using any of the methods described herein (e.g., using any of the detergents described herein, e.g., SDS and/or N-lauroylsarcosine sodium salt solution) before or after enzymatic treatment (e.g., treatment with any of the enzymes described herein, e.g., trypin, proteases (e.g., pepsin and/or proteinase K)).

In some instances, a biological sample can be obtained from non-mammalian organisms (e.g., a plants, an insect, an arachnid, a nematode (e.g., Caenorhabditis elegans), a fungi, an amphibian, or a fish (e.g., zebrafish)). In some instances, a biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. In some instances, biological samples can be derived from a homogeneous culture or population of the subjects or organisms mentioned herein or alternatively from a collection of several different organisms, for example, in a community or ecosystem.

In some instances, biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some instances, the biological sample is a cell culture sample. In some embodiments, the biological sample can be derived from a cell culture grown in vitro. Samples derived from a cell culture can include one or more suspension cells which are anchorage-independent within the cell culture.

As discussed above, a biological sample can include a single analyte of interest, or more than one analyte of interest. Methods for performing multiplexed assays to analyze two or more different analytes in a single biological sample is discussed in a subsequent section of this disclosure. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte can be an organelle (e.g., nuclei or mitochondria).

Examples of nucleic acid analytes include DNA analytes such as genomic DNA, methylated DNA, specific methylated DNA sequences, fragmented DNA, mitochondrial DNA, in situ synthesized PCR products, and RNA/DNA hybrids. Additional examples of nucleic acid analytes also include RNA analytes such as various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16 s rRNA or 23 s rRNA).

In general, the methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate. Methods for performing multiplexed assays to analyze two or more different analytes will be discussed in a subsequent section of this disclosure.

### (ii) Preparation of Biological Samples

A variety of steps can be performed to prepare a biological sample for analysis. Except where indicated otherwise, the preparative steps described below can generally be combined in any manner to appropriately prepare a particular sample for analysis.

In some instances, a biological sample can be harvested from a subject (e.g., via surgical biopsy, whole subject sectioning), grown in vitro on a growth substrate or culture dish as a population of cells, or prepared as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 micrometers thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 micrometers. Thicker sections can also be used if desired or convenient, e.g., at least 70, 80, 90, or 100 micrometers or more. Typically, the thickness of a tissue section is between 1-100 micrometers, 1-50 micrometers, 1-30 micrometers, 1-25 micrometers, 1-20 micrometers, 1-15 micrometers, 1-10 micrometers, 2-8 micrometers, 3-7 micrometers, or 4-6 micrometers, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

Additional methods can be performed on biological sample. For instances, in some embodiments, the biological sample (e.g., a tissue section as described above) can be prepared by deep freezing at a temperature suitable to maintain or preserve the integrity (e.g., the physical characteristics) of the tissue structure. Such a temperature can be, e.g., less than −20° C., or less than −25° C., −30° C., −40° C., −50° C., −60° C., −70° C., 80° C. −90° C., −100° C., −110° C., −120° C., −130° C., −140° C., −150° C., −160° C., −170° C., −180° C., −190° C., or −200° C.

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes). It is appreciated that other fixatives (e.g., ethanol, methanol, acetone, formaldehyde (e.g., 2% formaldehyde), paraformaldehyde-Triton, glutaraldehyde, or combinations thereof) can be used to fix the biological sample.

### B. Embedding and Partitioning of a Biological Sample

In some embodiments, methods provided herein include generating a plurality of partitions where a partition of the plurality of partitions includes a tissue section of the plurality of tissue sections. In general, each partition maintains separation of its own contents from the contents of other partitions. The partitions can be flowable within fluid streams. The partitions can be droplets of multiple phases. In some instances, the multiple phases include a first phase within a second phase, wherein the first and second phases are immiscible.

### (i) Biological Sample Embedding

In some embodiments, the polymer solution in methods provided herein is a hydrogel solution. The hydrogel solution can include a plurality of hydrogel subunits (e.g., hydrophilic monomers, molecular precursors, or polymers) that can be polymerized (e.g., cross-linked) to form a hydrogel matrix. In some embodiments, a biological sample (e.g., tissue section) is embedded in a hydrogel. In some embodiments, hydrogel subunits are infused into the biological sample, and polymerization of the hydrogel is initiated by an external or internal stimulus. A “hydrogel” as described herein can include a cross-linked 3D network of hydrophilic polymer chains. A “hydrogel subunit” can be a hydrophilic monomer, a molecular precursor, or a polymer that can be polymerized (e.g., cross-linked) to form a three-dimensional (3D) hydrogel network.

A hydrogel can swell in the presence of water. In some embodiments, a hydrogel comprises a natural material. In some embodiments, a hydrogel includes a synthetic material. In some embodiments, a hydrogel includes a hybrid material, e.g., the hydrogel material comprises elements of both synthetic and natural polymers. Any of the materials used in hydrogels or hydrogels comprising a polypeptide-based material described herein can be used. Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method known in the art. For example, the biological sample can be immobilized in the hydrogel by polyacrylamide crosslinking. Further, analytes of a biological sample can be immobilized in a hydrogel by crosslinking (e.g., polyacrylamide crosslinking).

The composition and application of the hydrogel to a biological sample typically depends on the nature and preparation of the biological sample (e.g., sectioned, non-sectioned, fresh-frozen tissue, type of fixation). A hydrogel can be any appropriate hydrogel where upon formation of the hydrogel on the biological sample the biological sample becomes anchored to or embedded in the hydrogel. Non-limiting examples of hydrogels are described herein or are known in the art. As one example, where the biological sample is a tissue section, the hydrogel can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel are formed in compartments, including but not limited to devices used to culture, maintain, or transport the cells. For example, hydrogels can be formed with monomer solution plus APS/TEMED added to the compartment to a depth ranging from about 0.1 μm to about 5 mm.

In some embodiments, a hydrogel includes a linker that allows anchoring of the biological sample to the hydrogel. In some embodiments, a hydrogel includes linkers that allow anchoring of biological analytes to the hydrogel. In such cases, the linker can be added to the hydrogel before, contemporaneously with, or after hydrogel formation. Non-limiting examples of linkers that anchor nucleic acids to the hydrogel can include 6-((Acryloyl)amino) hexanoic acid (Acryloyl-X SE) (available from ThermoFisher, Waltham, MA), Label-IT Amine (available from MirusBio, Madison, WI) and Label X (Chen et al., Nat. Methods 13:679-684, (2016)).

In some embodiments, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., capture probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell tagging agent is added to the hydrogel before, contemporaneously with, and/or after polymerization. In some embodiments, a cell-penetrating agent is added to the hydrogel before, contemporaneously with, and/or after polymerization.

In some embodiments, a biological sample is embedded in a hydrogel to facilitate sample transfer to another location (e.g., to an array). For example, archived biological samples (e.g., FFPE tissue sections) can be transferred from storage to a spatial array to perform spatial analysis. In some embodiments, a biological sample on a substrate can be covered with any of the prepolymer solutions described herein. In some embodiments, the prepolymer solution can be polymerized such that a hydrogel is formed on top of and/or around the biological sample. Hydrogel formation can occur in a manner sufficient to anchor (e.g., embed) the biological sample to the hydrogel. After hydrogel formation, the biological sample is anchored to (e.g., embedded in) the hydrogel wherein separating the hydrogel from the substrate (e.g., glass slide) results in the biological sample separating from the substrate along with the hydrogel. The biological sample contained in the hydrogel can then be contacted with a spatial array, and spatial analysis can be performed on the biological sample.

Any variety of characteristics can determine the transfer conditions required for a given biological sample. Non-limiting examples of characteristics likely to impact transfer conditions include the sample (e.g., thickness, fixation, and cross-linking) and/or the analyte of interest (different conditions to preserve and/or transfer different analytes (e.g., DNA, RNA, and protein)).

In some embodiments, the hydrogel is removed after contacting the biological sample with the spatial array. For example, methods described herein can include an event-dependent (e.g., light or chemical) depolymerizing hydrogel, wherein upon application of the event (e.g., external stimuli) the hydrogel depolymerizes. In one example, a biological sample can be anchored to a DTT-sensitive hydrogel, where addition of DTT can cause the hydrogel to depolymerize and release the anchored biological sample.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored in a medium before or after clearing of hydrogel (e.g., a mounting medium, methylcellulose, or other semi-solid mediums).

In some embodiments, the hydrogel chemistry can be tuned to specifically bind (e.g., retain) particular species of analytes (e.g., RNA, DNA, protein, etc.). In some embodiments, a hydrogel includes a linker that allows anchoring of the biological sample to the hydrogel. In some embodiments, a hydrogel includes linkers that allow anchoring of biological analytes to the hydrogel. In such cases, the linker can be added to the hydrogel before, contemporaneously with, or after hydrogel formation. Non-limiting examples of linkers that anchor nucleic acids to the hydrogel can include 6-((Acryloyl) amino) hexanoic acid (Acryloyl-X SE), Label-IT Amine and Label X (Chen et al., Nat. Methods 13:679-684, (2016)). Non-limiting examples of characteristics likely to impact transfer conditions include the sample (e.g., thickness, fixation, and cross-linking) and/or the analyte of interest (different conditions to preserve and/or transfer different analytes (e.g., DNA, RNA, and protein)).

Additional methods and aspects of hydrogel embedding of biological samples are described for example in Chen et al., Science 347(6221):543-548, 2015, the entire contents of which are incorporated herein by reference.

In some instances, methods involving use of a hydrogel can include forming a hydrogel matrix from the hydrogel solution, and generating a plurality of hydrogel beads (e.g., called “macrobeads” in some instances), where a hydrogel macrobead of the plurality of hydrogel macrobeads includes a biological (e.g., tissue) section of the plurality of tissue sections.

In some instances, forming a hydrogel matrix from the hydrogel solution immobilizes or anchors the plurality of tissue sections within the hydrogel matrix. The hydrogel matrix can then be dissociated into a plurality of chunks, where a chunk of the plurality of chunks includes a tissue section of the plurality of tissue sections. The chunks can be formed into various shapes and/or dimensions depending on the context of the intended use. In some instances, the chunks are shaped into beads (e.g., macrobeads). The volume, fluidity, porosity, and/or rigidity of the beads can be dependent on the type of materials used to form the hydrogel matrix. The hydrogel matrix can be dissociated into chunks by various mechanical means (e.g., cutting, stamping, microdissecting), acoustic means (e.g., sonication), or any other suitable method provided herein.

A hydrogel macrobead can contain one or more tissue sections. In some instances, a hydrogel macrobead contains a single tissue section. In instances where a hydrogel macrobead contains more than one tissue sections, each tissue section can be physically separated from other tissue sections in the same macrobead. The size of a hydrogel macrobead can be dependent on the size and/or thickness of the tissue sections, and can be adapted to fully encapsulate one or more tissue sections. The macrobeads can be of uniform size or heterogeneous size.

### (ii) Biological Sample Partitioning

In some embodiments, generating a plurality of partitions includes generating a plurality of droplets containing the polymer solution, where a droplet of the plurality of droplets includes a tissue section of the plurality of tissue sections. The partitions can be droplets of aqueous fluid within a non-aqueous continuous phase (e.g., oil phase). In another example, the partitions can be droplets of a non-aqueous fluid within an aqueous phase. The plurality of droplets can be generated using any of the droplet generating methods provided herein. For instance, droplets can be formed in an emulsion that contains a second solution. For droplets in an emulsion, allocating individual tissue sections to discrete partitions can be accomplished, for example, by introducing a flowing stream of tissue sections in an aqueous fluid into a flowing stream of a non-aqueous fluid, such that droplets are generated at the junction of the two streams. In some instances, the partitions are provided in a water-in-oil emulsion or oil-in-water emulsion. In some instances, the droplets are generated in a microfluidic device.

A droplet can contain one or more tissue sections. In some instances, a droplet contains a single tissue section. The size of a droplet can be dependent on the size and/or thickness of the tissue sections, and can be adapted to fully encapsulate one or more tissue sections. The average volume of droplets in the plurality of droplets can be less than 10,000 picoliters (e.g., less than 8000, 7000, 6000, 5000, 4000, 3000, 2000, 1000, or 800 picoliters).

In some instances, the biological sample is partitioned into serial sections of the same sample (e.g., tissue). In some embodiments, the biological sample can optionally be separated into single cells, cell groups, or other fragments/pieces that are smaller than the original, unfragmented sample. Each of these smaller portions of the sample can be analyzed to obtain spatially-resolved analyte information for the sample.

For samples that have been separated into smaller fragments-and particularly, for samples that have been disaggregated, dissociated, or otherwise separated into individual cells-one method for analyzing the fragments involves separating the fragments into individual partitions (e.g., fluid droplets), and then analyzing the contents of the partitions. In general, each partition maintains separation of its own contents from the contents of other partitions. The partition can be a droplet in an emulsion, for example.

The partitions can be flowable within fluid streams. The partitions can include, for example, micro-vesicles that have an outer barrier surrounding an inner fluid center or core. In some cases, the partitions can include a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions can be provided in a water-in-oil emulsion or oil-in-water emulsion. A variety of different vessels are described in, for example, U.S. Patent Application Publication No. 2014/0155295, the entire contents of which are incorporated herein by reference. Emulsion systems for creating stable droplets in non-aqueous or oil continuous phases are described, for example, in U.S. Patent Application Publication No. 2010/0105112, the entire contents of which are incorporated herein by reference.

For droplets in an emulsion, allocating individual particles to discrete partitions can be accomplished, for example, by introducing a flowing stream of particles in an aqueous fluid into a flowing stream of a non-aqueous fluid, such that droplets are generated at the junction of the two streams. Fluid properties (e.g., fluid flow rates, fluid viscosities, etc.), particle properties (e.g., volume fraction, particle volume, particle concentration, etc.), microfluidic architectures (e.g., channel geometry, etc.), and other parameters can be adjusted to control the occupancy of the resulting partitions (e.g., number of analytes per partition, number of beads per partition, etc.). For example, partition occupancy can be controlled by providing the aqueous stream at a certain concentration and/or flow rate of analytes.

### C. Manipulation of the Biological Sample

### (i) Manipulation of Biological Sample in Solution

In addition to cells and/or analytes, a partition can include additional components, and in particular, one or more beads. A partition can include a single gel bead, a single cell bead, or both a single cell bead and single gel bead. A variety of different beads can be incorporated into partitions. In some embodiments, for example, non-barcoded beads can be incorporated into the partitions. For example, where the biological particle (e.g., a cell) that is incorporated into the partitions carries one or more barcodes (e.g., spatial barcode(s), UMI(s), and combinations thereof), the bead can be a non-barcoded bead.

In some embodiments, a barcode carrying bead can be incorporated into partitions. In general, an individual bead can be coupled to any number of individual nucleic acid molecules, for example, from one to tens to hundreds of thousands or even millions of individual nucleic acid molecules. The respective barcodes for the individual nucleic acid molecules can include both common sequence segments or relatively common sequence segments and variable or unique sequence segments between different individual nucleic acid molecules coupled to the same bead. For example, a nucleic acid molecule (e.g., an oligonucleotide), can be coupled to a bead by a releasable linkage (e.g., a disulfide linker), wherein the nucleic acid molecule can be or include a barcode. For example, barcodes can be injected into droplets previous to, subsequent to, or concurrently with droplet generation. The delivery of the barcodes to a particular partition allows for the later attribution of the characteristics of the individual biological particle to the particular partition. Barcodes can be delivered, for example on a nucleic acid molecule (e.g., an oligonucleotide), to a partition via any suitable mechanism. Barcoded nucleic acid molecules can be delivered to a partition via a microcapsule. A microcapsule, in some instances, can include a bead. The same bead can be coupled (e.g., via releasable linkage) to one or more other nucleic acid molecules.

In some embodiments, a microcapillary array with spatially barcoded beads can be generated. A plurality of spatially barcoded beads can be flowed into channels on a microcapillary array such that each microcapillary channel can be loaded with one spatially barcoded bead. In some embodiments, the spatially barcoded bead microcapillary array can be contacted to a biological sample for subsequent spatial analysis of biological analytes within the biological sample. In some embodiments, a microcapillary array channel can mechanically compress the biological sample and form fluidically isolated reaction chambers. In some embodiments, reagents (e.g., enzymes, nucleic acids) are introduced into the reaction chambers. The reagents can be sealed (e.g., by silicone oil, mineral oil) within the reaction chambers and incubated, allowing for a cellular and/or nuclear permeabilization reaction to occur. In some embodiments, biological analytes (e.g., DNA, RNA, proteins, metabolites, small molecules, and lipids) are released and captured onto the spatially barcoded microcapillary array, preserving their spatial information. In some embodiments, spatial analysis using a spatially barcoded feature microcapillary array can be used to obtain spatial information of the biological sample analytes at single-cell resolution.

The nucleic acid molecule can include a functional domain that can be used in subsequent processing. For example, the functional domain can include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule can include a barcode sequence for use in barcoding the sample (e.g., DNA, RNA, protein, etc.). In some cases, the barcode sequence can be bead-specific such that the barcode sequence is common to all nucleic acid molecules coupled to the same bead. Alternatively or in addition, the barcode sequence can be partition-specific such that the barcode sequence is common to all nucleic acid molecules coupled to one or more beads that are partitioned into the same partition. The nucleic acid molecule can include a specific priming sequence, such as an mRNA specific priming sequence (e.g., poly(T) sequence), a targeted priming sequence, and/or a random priming sequence. The nucleic acid molecule can include an anchoring sequence to ensure that the specific priming sequence hybridizes at the sequence end (e.g., of the mRNA). For example, the anchoring sequence can include a random short sequence of nucleotides, such as a 1-mer, 2-mer, 3-mer or longer sequence, which can ensure that a poly(T) segment is more likely to hybridize at the sequence end of the poly(A) tail of the mRNA.

The nucleic acid molecule can include a unique molecular identifying sequence (e.g., unique molecular identifier (UMI)). In some embodiments, the unique molecular identifying sequence can include from about 5 to about 8 nucleotides. Alternatively, the unique molecular identifying sequence can include less than about 5 or more than about 8 nucleotides. The unique molecular identifying sequence can be a unique sequence that varies across individual nucleic acid molecules coupled to a single bead. In some embodiments, the unique molecular identifying sequence can be a random sequence (e.g., such as a random N-mer sequence). For example, the UMI can provide a unique identifier of the starting mRNA molecule that was captured, in order to allow quantitation of the number of original expressed RNA.

A partition can also include one or more reagents. Unique identifiers, such as barcodes, can be injected into the droplets previous to, subsequent to, or concurrently with droplet generation, such as via a microcapsule (e.g., bead). Microfluidic channel networks (e.g., on a chip) can be utilized to generate partitions. Alternative mechanisms can also be employed in the partitioning of individual biological particles, including porous membranes through which aqueous mixtures of cells are extruded into non-aqueous fluids.

In some embodiments, barcoded nucleic acid molecules can be initially associated with a microcapsule and then released from the microcapsule. Release of the barcoded nucleic acid molecules can be passive (e.g., by diffusion out of the microcapsule). In addition or alternatively, release from the microcapsule can be upon application of a stimulus which allows the barcoded nucleic acid nucleic acid molecules to dissociate or to be released from the microcapsule. Such stimulus can disrupt the microcapsule, an interaction that couples the barcoded nucleic acid molecules to or within the microcapsule, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

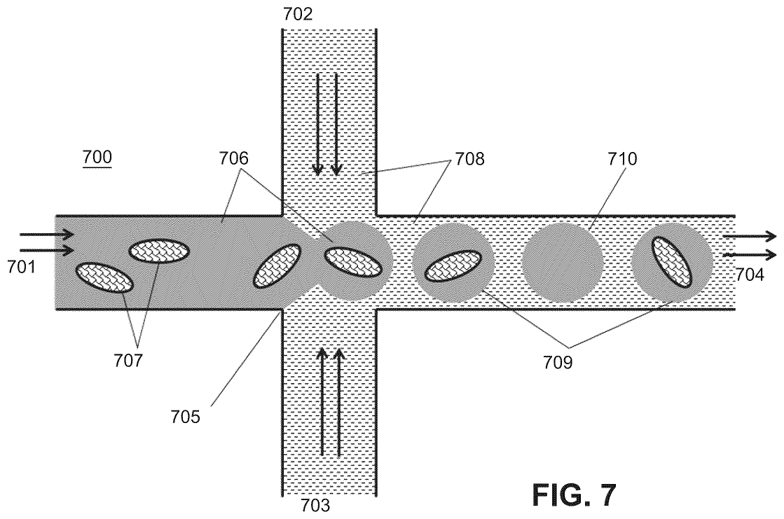

In some embodiments, one more barcodes (e.g., spatial barcodes, UMIs, or a combination thereof) can be introduced into a partition as part of the analyte. As described previously, barcodes can be bound to the analyte directly, or can form part of a capture probe or analyte capture agent that is hybridized to, conjugated to, or otherwise associated with an analyte, such that when the analyte is introduced into the partition, the barcode(s) are introduced as well. As described above, FIG. 7 shows an example of a microfluidical channel structure for partitioning individual analytes (e.g., cells, tissue sections, or tissue section containing beads or droplets) into discrete partitions.

FIG. 7 shows an example of a microfluidic channel structure for partitioning individual analytes (e.g., cells, tissue sections, or tissue section containing beads or droplets) into discrete partitions. The channel structure can include channel segments 701, 702, 703, and 704 communicating at a channel junction 705. In operation, a first aqueous fluid 706 that includes suspended biological particles (or cells, tissue sections, or tissue section containing beads or droplets, etc.) 707 may be transported along channel segment 701 into junction 705, while a second fluid 708 that is immiscible with the aqueous fluid 706 is delivered to the junction 705 from each of channel segments 702 and 703 to create discrete droplets 709, 710 of the first aqueous fluid 706 flowing into channel segment 704, and flowing away from junction 705. The channel segment 704 may be fluidically coupled to an outlet reservoir where the discrete droplets can be stored and/or harvested. A discrete droplet generated may include an individual biological particle 707 (such as droplets 709). A discrete droplet generated may include more than one individual biological particle 707. A discrete droplet may contain no biological particle 707 (such as droplet 710). Each discrete partition may maintain separation of its own contents (e.g., individual biological particle 707) from the contents of other partitions.

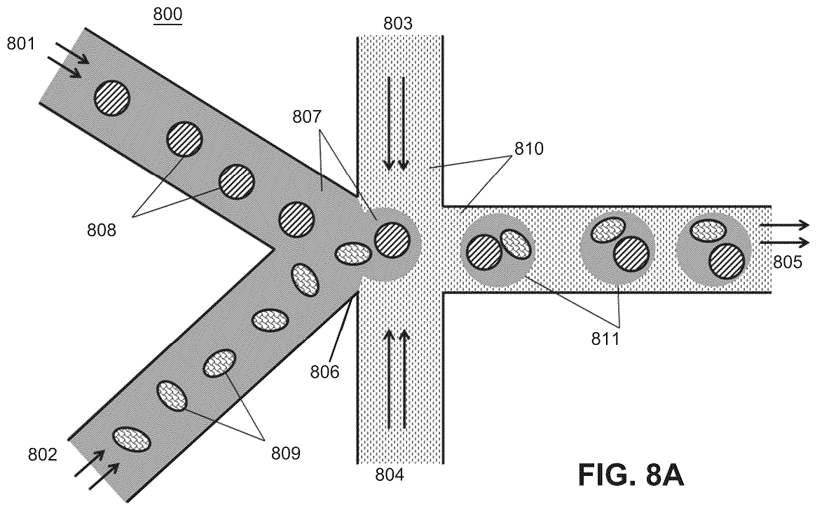

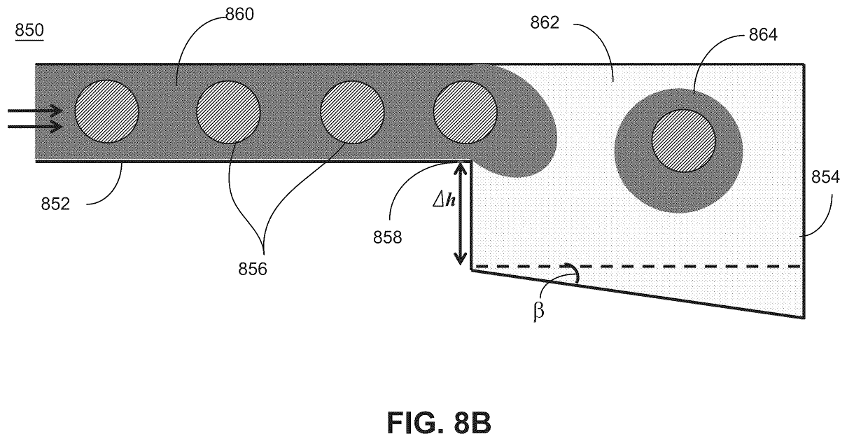

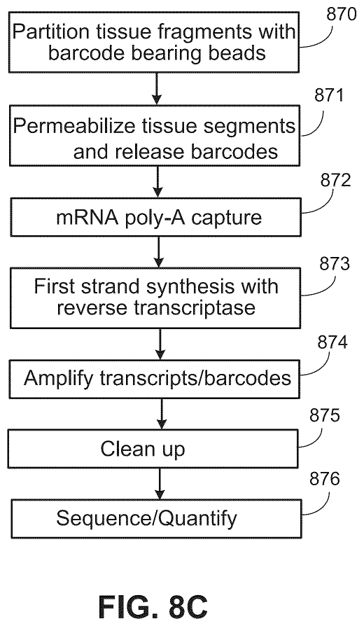

FIG. 8A shows another example of a microfluidic channel structure 800 for delivering tissue section containing beads and additional beads with reagents, etc. to droplets. The channel structure includes channel segments 801, 802, 803, 804 and 805 communicating at a channel junction 806. During operation, the channel segment 801 can transport an aqueous fluid 807 that includes a plurality of beads 808 along the channel segment 801 into junction 806. The plurality of beads 808 can be sourced from a suspension of beads. For example, the channel segment 801 can be connected to a reservoir that includes an aqueous suspension of beads 808. The channel segment 802 can transport the aqueous fluid 807 that includes a plurality of particles 809 (e.g., cells, tissues, etc.) along the channel segment 802 into junction 806. In some embodiments, the aqueous fluid 807 in either the first channel segment 801 or the second channel segment 802, or in both segments, can include one or more reagents, as further described below.

A second fluid 810 that is immiscible with the aqueous fluid 807 (e.g., oil) can be delivered to the junction 806 from each of channel segments 803 and 804. Upon meeting of the aqueous fluid 807 from each of channel segments 801 and 802 and the second fluid 810 from each of channel segments 803 and 804 at the channel junction 806, the aqueous fluid 807 can be partitioned as discrete droplets 811 in the second fluid 810 and flow away from the junction 806 along channel segment 805. The channel segment 805 can deliver the discrete droplets to an outlet reservoir fluidly coupled to the channel segment 805, where they can be harvested.

As an alternative, the channel segments 801 and 802 can meet at another junction upstream of the junction 806. At such junction, beads and biological particles can form a mixture that is directed along another channel to the junction 806 to yield droplets 811. The mixture can provide the beads and biological particles in an alternating fashion, such that, for example, a droplet includes a single bead and a single biological particle, such as a tissue fragment.

The second fluid 810 can include an oil, such as a fluorinated oil, that includes a fluorosurfactant for stabilizing the resulting droplets, for example, inhibiting subsequent coalescence of the resulting droplets 811.

The partitions described herein can include small volumes, for example, less than about 10 microliters (TL), 5 TL, 1 TL, 900 picoliters (pL), 800 pL, 700 pL, 600 pL, 500 pL, 400 pL, 300 pL, 200 pL, 100pL, 50 pL, 20 pL, 10 pL, 1 pL, 500 nanoliters (nL), 100 nL, 50 nL, or less. In the foregoing discussion, droplets with beads were formed at the junction of different fluid streams. In some embodiments, droplets can be formed by gravity-based partitioning methods.

FIG. 8B shows a cross-section view of another example of a microfluidic channel structure 850 with a geometric feature for controlled partitioning. A channel structure 850 can include a channel segment 852 communicating at a channel junction 858 (or intersection) with a reservoir 854. In some instances, the channel structure 850 and one or more of its components can correspond to the channel structure 800 and one or more of its components.

An aqueous fluid 860 comprising a plurality of particles 856 may be transported along the channel segment 852 into the junction 858 to meet a second fluid 862 (e.g., oil, etc.) that is immiscible with the aqueous fluid 860 in the reservoir 854 to create droplets 864 of the aqueous fluid 860 flowing into the reservoir 854. At the junction 858 where the aqueous fluid 860 and the second fluid 862 meet, droplets can form based on factors such as the hydrodynamic forces at the junction 858, relative flow rates of the two fluids 860, 862, fluid properties, and certain geometric parameters (e.g., Δh, etc.) of the channel structure 850. A plurality of droplets can be collected in the reservoir 854 by continuously injecting the aqueous fluid 860 from the channel segment 852 at the junction 858.

A discrete droplet generated may comprise one or more particles of the plurality of particles 856. As described elsewhere herein, a particle may be any particle, such as a bead, cell bead, gel bead, biological particle, macromolecular constituents of biological particle, or other particles. Alternatively, a discrete droplet generated may not include any particles.

In some instances, the aqueous fluid 860 can have a substantially uniform concentration or frequency of particles 856. As described elsewhere herein, the particles 856 (e.g., beads) can be introduced into the channel segment 852 from a separate channel (not shown in FIGS. 8A-8B). The frequency of particles 856 in the channel segment 852 may be controlled by controlling the frequency in which the particles 856 are introduced into the channel segment 852 and/or the relative flow rates of the fluids in the channel segment 852 and the separate channel. In some instances, the particles 856 can be introduced into the channel segment 852 from a plurality of different channels, and the frequency controlled accordingly. In some instances, different particles may be introduced via separate channels. For example, a first separate channel can introduce beads and a second separate channel can introduce biological particles into the channel segment 852. The first separate channel introducing the beads may be upstream or downstream of the second separate channel introducing the biological particles.

In some instances, the second fluid 862 may not be subjected to and/or directed to any flow in or out of the reservoir 854. For example, the second fluid 862 may be substantially stationary in the reservoir 854. In some instances, the second fluid 862 may be subjected to flow within the reservoir 854, but not in or out of the reservoir 854, such as via application of pressure to the reservoir 854 and/or as affected by the incoming flow of the aqueous fluid 860 at the junction 858. Alternatively, the second fluid 862 may be subjected and/or directed to flow in or out of the reservoir 854. For example, the reservoir 854 can be a channel directing the second fluid 862 from upstream to downstream, transporting the generated droplets.

The channel structure 850 at or near the junction 858 may have certain geometric features that at least partly determine the volumes and/or shapes of the droplets formed by the channel structure 850. The channel segment 852 can have a first cross-section height, h1, and the reservoir 854 can have a second cross-section height, h2. The first cross-section height, h1, and the second cross-section height, h2, may be different, such that at the junction 858, there is a height difference of Δh. The second cross-section height, h2, may be greater than the first cross-section height, h1. In some instances, the reservoir may thereafter gradually increase in cross-section height, for example, the more distant it is from the junction 858. In some instances, the cross-section height of the reservoir may increase in accordance with expansion angle, β, at or near the junction 858. The height difference, Δh, and/or expansion angle, β, can allow the tongue (portion of the aqueous fluid 860 leaving channel segment 852 at junction 858 and entering the reservoir 854 before droplet formation) to increase in depth and facilitate decrease in curvature of the intermediately formed droplet. For example, droplet volume may decrease with increasing height difference and/or increasing expansion angle.

The height difference, Δh, can be at least about 1 μm. Alternatively, the height difference can be at least about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500 μm or more. Alternatively, the height difference can be at most about 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1 μm or less. In some instances, the expansion angle, B, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less.

In some instances, the flow rate of the aqueous fluid 860 entering the junction 858 can be between about 0.04 microliters (μL)/minute (min) and about 40 μL/min. In some instances, the flow rate of the aqueous fluid 860 entering the junction 858 can be between about 0.01 microliters (μL)/minute (min) and about 100 μL/min. Alternatively, the flow rate of the aqueous fluid 860 entering the junction 858 can be less than about 0.01 μL/min. alternatively, the flow rate of the aqueous fluid 860 entering the junction 858 can be greater than about 40 μL/min, such as 45 μL/min, 50 μL/min, 55 μL/min, 60 μL/min, 65 μL/min, 70 μL/min, 75 μL/min, 80 μL/min, 85 L/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 860 entering the junction 858. The second fluid 862 may be stationary, or substantially stationary, in the reservoir 854. Alternatively, the second fluid 862 may be flowing, such as at the above flow rates described for the aqueous fluid 860.

While FIG. 8B illustrates the height difference, Δh, being abrupt at the junction 858 (e.g., a step increase), the height difference may increase gradually (e.g., from about 0 μm to a maximum height difference). Alternatively, the height difference may decrease gradually (e.g., taper) from a maximum height difference. A gradual increase or decrease in height difference, as used herein, may refer to a continuous incremental increase or decrease in height difference, wherein an angle between any one differential segment of a height profile and an immediately adjacent differential segment of the height profile is greater than 90°. For example, at the junction 858, a bottom wall of the channel and a bottom wall of the reservoir can meet at an angle greater than 90°. Alternatively or in addition, a top wall (e.g., ceiling) of the channel and a top wall (e.g., ceiling) of the reservoir can meet an angle greater than 90°. A gradual increase or decrease may be linear or non-linear (e.g., exponential, sinusoidal, etc.). Alternatively or in addition, the height difference may variably increase and/or decrease linearly or non-linearly. While FIG. 8B illustrates the expanding reservoir cross-section height as linear (e.g., constant expansion angle, B), the cross-section height may expand non-linearly. For example, the reservoir may be defined at least partially by a dome-like (e.g., hemispherical) shape having variable expansion angles. The cross-section height may expand in any shape.

FIG. 8C depicts a workflow wherein tissue sections are partitioned into droplets along with barcode-bearing beads 870. See FIG. 8A. The droplet forms an isolated reaction chamber wherein the tissue sections can be permeabilized and/or cells within the tissue sections can be lysed 871 and target analytes within the tissue fragments can then be captured 872 and amplified 873, 874 according to previously described methods. After sequence library preparation clean-up 875, the material is sequenced and/or quantified 876 according to methods described herein. For example, the workflow shown in FIG. 8C can be used with a biological sample on an array, where the features of the array have been delivered to the substrate via a droplet manipulation system. In some embodiments, capture probes on the features can specifically bind analytes present in the biological sample. In some embodiments, the features can be removed from the substrate (e.g., removed by any method described herein) and partitioned into droplets with barcode-bearing beads for further analysis according to methods described herein.

It should be noted that while the example workflow in FIG. 8C includes steps specifically for the analysis of mRNA, analogous workflows can be implemented for a wide variety of other analytes, including any of the analytes described previously.

By way of example, in the context of analyzing sample RNA as shown in FIG. 8C, the poly(T) segment of one of the released nucleic acid molecules (e.g., from the bead) can hybridize to the poly(A) tail of an mRNA molecule. Reverse transcription can result in a cDNA transcript of the mRNA, which transcript includes each of the sequence segments of the nucleic acid molecule. If the nucleic acid molecule includes an anchoring sequence, it will more likely hybridize to and prime reverse transcription at the sequence end of the poly(A) tail of the mRNA.

Within any given partition, all of the cDNA transcripts of the individual mRNA molecules can include a common barcode sequence segment. However, the transcripts made from the different mRNA molecules within a given partition can vary at the unique molecular identifying sequence segment (e.g., UMI segment). Beneficially, even following any subsequent amplification of the contents of a given partition, the number of different UMIs can be indicative of the quantity of mRNA originating from a given partition. As noted above, the transcripts can be amplified, cleaned up and sequenced to identify the sequence of the cDNA transcript of the mRNA, as well as to sequence the barcode segment and the UMI segment. While a poly(T) primer sequence is described, other targeted or random priming sequences can also be used in priming the reverse transcription reaction. Likewise, although described as releasing the barcoded oligonucleotides into the partition, in some cases, the nucleic acid molecules bound to the bead can be used to hybridize and capture the mRNA on the solid phase of the bead, for example, in order to facilitate the separation of the RNA from other cell contents.

In some embodiments, partitions include precursors that include a functional group that is reactive or capable of being activated such that it becomes reactive can be polymerized with other precursors to generate gel beads that include the activated or activatable functional group. The functional group can then be used to attach additional species (e.g., disulfide linkers, primers, other oligonucleotides, etc.) to the gel beads. For example, some precursors featuring a carboxylic acid (COOH) group can co-polymerize with other precursors to form a bead that also includes a COOH functional group. In some cases, acrylic acid (a species comprising free COOH groups), acrylamide, and bis(acryloyl) cystamine can be co-polymerized together to generate a bead with free COOH groups. The COOH groups of the bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

In some embodiments, a bead can be formed from materials that include degradable chemical cross-linkers, such as BAC or cystamine. Degradation of such degradable cross-linkers can be accomplished through a number of mechanisms. In some examples, a bead can be contacted with a chemical degrading agent that can induce oxidation, reduction or other chemical changes. For example, a chemical degrading agent can be a reducing agent, such as dithiothreitol (DTT). Additional examples of reducing agents can include β-mercaptoethanol, (2S)-2-amino-1,4-dimercaptobutane (dithiobutylamine or DTBA), tris(2-carboxyethyl) phosphine (TCEP), or combinations thereof. A reducing agent can degrade the disulfide bonds formed between gel precursors forming the bead, and thus, degrade the bead.

In some embodiments, beads that are partitioned with the analyte can include different types of oligonucleotides bound to the bead, where the different types of oligonucleotides bind to different types of analytes. For example, a bead can include one or more first oligonucleotides (which can be capture probes, for example) that can bind or hybridize to a first type of analyte, such as mRNA for example, and one or more second oligonucleotides (which can be capture probes, for example) that can bind or hybridize to a second type of analyte, such as gDNA for example. Partitions can also include lysis agents that aid in releasing nucleic acids from the co-partitioned cell, and can also include an agent (e.g., a reducing agent) that can degrade the bead and/or break covalent linkages between the oligonucleotides and the bead, releasing the oligonucleotides into the partition. The released barcoded oligonucleotides (which can also be barcoded) can hybridize with mRNA released from the cell and also with gDNA released from the cell.

Barcoded constructs thus formed from hybridization can include a first type of construct that includes a sequence corresponding to an original barcode sequence from the bead and a sequence corresponding to a transcript from the cell, and a second type of construct that includes a sequence corresponding to the original barcode sequence from the bead and a sequence corresponding to genomic DNA from the cell. The barcoded constructs can then be released/removed from the partition and, in some embodiments, further processed to add any additional sequences. The resulting constructs can then be sequenced, the sequencing data processed, and the results used to spatially characterize the mRNA and the gDNA from the cell.

In another example, a partition includes a bead that includes a first type of oligonucleotide (e.g., a first capture probe) with a first barcode sequence, a poly(T) capture sequence that can hybridize with the poly (A) tail of an mRNA transcript, and a UMI barcode sequence that can uniquely identify a given transcript. The bead also includes a second type of oligonucleotide (e.g., a second capture probe) with a second barcode sequence, a targeted priming sequence that is capable of specifically hybridizing with a third barcoded oligonucleotide (e.g., an analyte capture agent) coupled to an antibody that is bound to the surface of the partitioned cell. The third barcoded oligonucleotide includes a UMI barcode sequence that uniquely identifies the antibody (and thus, the particular cell surface feature to which it is bound).

In some instances, a partition described herein further includes one or more beads (e.g. any of the beads described herein) that can be delivered into the partition via any of the methods provided herein. The beads can be barcoded or non-barcoded. In some instances, the beads include a capture probe (e.g., any of the capture probes described herein), which can bind to a biological analyte (e.g., any of the biological analytes described herein) in the tissue section within the partition. In some instances, a partition of the plurality of partitions described herein further includes an analyte capture agent capable of binding to a biological analyte in the tissue section within the partition. Analysis of the captured analytes can be carried out according to any of the methods provided herein. Multiple analytes in a tissue section within a partition can be analyzed in parallel. For example, a bead with capture probes capable of binding to different biological analytes can be included in the partition. In some embodiments, the analytes are of the same type (e.g., the analytes can be RNA, e.g., mRNAs). In some embodiments, the analytes are of two or more types (e.g., RNA, DNA, and/or proteins can be analyzed in parallel).

The partitions can also include additional agents, such as but not limited to, lysis agents that aid in releasing biological analytes from the tissue section, DNase and RNase inactivating agents or inhibitors, such as proteinase K, chelating agents, such as EDTA, and/or other reagents employed in removing or otherwise reducing negative activity or impact of different cell lysate components on subsequent processing of nucleic acids. Additionally or alternatively, reagents that can also be co-partitioned include, without limitation, endonucleases to fragment DNA, DNA polymerase enzymes and/or dNTPs used to amplify nucleic acid fragments and to attach the barcode molecular tags to the amplified fragments. Additionally or alternatively, reagents that can also be co-partitioned include, without limitation, reverse transcriptase enzymes, including enzymes with terminal transferase activity, primers and oligonucleotides, and/or switch oligonucleotides (also referred to herein as “switch oligos” or “template switching oligonucleotides”) which can be used for template switching.

In some instances, methods provided herein include staining the tissue section using any of the suitable staining techniques described herein or known in the art (e.g., IHC, IF, or chemical staining). For instance, a partition containing a tissue section can be deposited into one or more staining solutions, where the tissue section is exposed to staining reagents. The partition can be incubated in the staining solutions for a period of time sufficient to allow the staining agent to contact and stain the tissue section. A plurality of partitions can be processed together. For instance, multiple partitions can be deposited into the same staining solution at one time. In some instances, at least 10 (e.g., at least 20, 50, 80, 120, 160, 200, 300, or 500) partitions can be processed in parallel.

Staining the tissue section in a partition can include labeling biological analytes in the tissue section with a detectable label (e.g., an optical label). Any suitable optical labels can be used in methods provided herein, including but not limited to, fluorescent, radioactive, chemiluminescent, calorimetric, and/or colorimetric detectable labels. In some instances, methods provided herein include staining the tissue section and labeling two or more (e.g., 3 or more, 5 or more, 8 or more, or 12 or more) biological analytes in the tissue section with an optical label. The biological analytes can be any suitable biological analytes described herein, e.g., RNA, DNA, or protein. The material encapsulating the tissue section within a partition can be substantially transparent to allow detection of the optical labels attached to the biological analytes in the tissue section.

### (ii) Microscopy and Analysis

In some instances, methods provided herein further include imaging the tissue section within a partition. The tissue section can be removed from the partition prior to imaging, or can be retained in the partition for imaging. Any suitable imaging techniques described herein are contemplated, including brightfield and fluorescence modalities, using a variety of different techniques, e.g., expansion microscopy, bright field microscopy, dark field microscopy, phase contrast microscopy, electron microscopy, fluorescence microscopy, reflection microscopy, interference microscopy, confocal microscopy, and visual identification (e.g., by eye), and combinations thereof. In some instances, the partitions are subjected to capillary microscopy. In some instances, the partitions are subjected to FACS sorting.

Methods provided herein can further include, prior to imaging the tissue section, depositing the partition containing the tissue section on a substrate (e.g., any of the variety of substrates described herein). For instance, the substrate can be a multi-well plate where one well is configured to accommodate one partition. The substrate can be a glass slide, where one partition is deposited on one slide. The substrate can be positively charged to aid in attachment of the partition. In some instances, the partition is attached to the substrate via a chemical linker. The partition can be attached to the substrate reversibly or irreversibly, depending upon the nature of the partition and subsequent steps in the analytical method. In some instances, upon placement on the substrate having a flat surface, the tissue section within the partition unfolds to be substantially flat.

The material encapsulating the tissue section can be removed after the partition is deposited on the substrate. In some embodiments, removing the material encapsulating the tissue section facilitates unfolding of the tissue section onto the substrate and/or in imaging analysis. As an example, for hydrogel macrobeads containing tissue sections, the hydrogel can be removed after the macrobeads are deposited onto the substrate. The hydrogel macrobeads described herein can include an event-dependent (e.g., light-dependent or chemical-dependent) depolymerizing hydrogel, wherein upon application of the event (e.g., external stimuli) the hydrogel depolymerizes. In some examples, the hydrogel macrobeads can include a DTT-sensitive hydrogel, where addition of DTT can cause the hydrogel to depolymerize and release the tissue section.

Upon depositing the partition on a substrate, the methods may further include applying heat to the substrate, e.g., facilitating the tissue section to unfold onto the substrate for imaging analysis. Heat can be applied to the entire substrate or portions of the substrate. For instance, heat can be applied to a portion of the substrate corresponding to the location of the partition, e.g., directly underneath the partition.

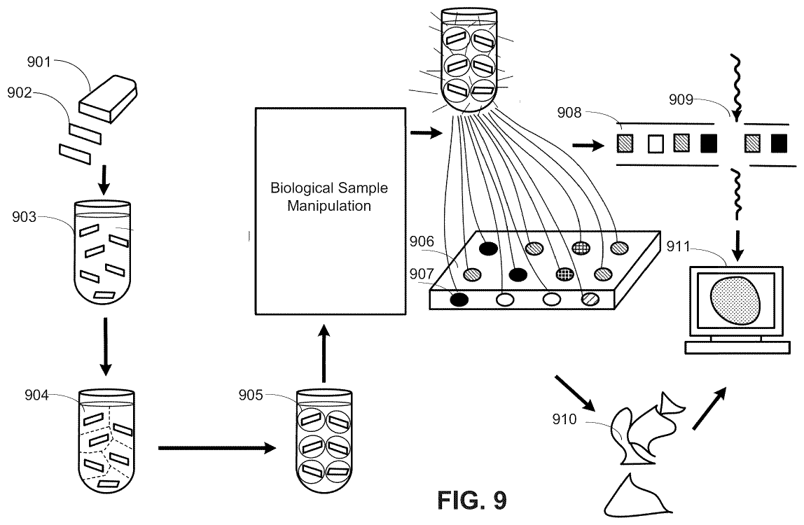

FIG. 9 is a workflow schematic illustrating exemplary steps of generating tissue macrobeads or droplets for various types of imaging analysis.

### D. Compositions and Kits

In some embodiments, also provided herein are kits that include one or more reagents to prepare a spatial array as described herein. In some instances, the kit includes a polymer solution comprising a hydrogel; a container for the polymer solution; one or more non-aqueous droplets; one or more compositions to manipulate the biological sample; and instructions to perform the methods disclosed herein. In some embodiments, the kits can include one or more enzymes for performing any of the methods described herein, including but not limited to, a DNA polymerase, a reverse transcriptase, a ligase, an endonuclease, a protease, or a combination thereof.

In some embodiments, a non-limiting example of a kit used to perform any of the methods described herein includes: (a) a polymer solution comprising a hydrogel; (b) a container for the polymer solution; (c) one or more non-aqueous droplets to partition a biological sample comprising an analyte; (d) one or more compositions to manipulate the biological sample, wherein the one or more compositions are selected from the group consisting of: (i) a plurality of capture probes, wherein a capture probe of the plurality comprises (i) a spatial barcode and (ii) a capture domain that binds to a sequence present in the analyte; (ii) a plurality of analyte capture agents, wherein an analyte capture agent of the plurality of analyte capture agents comprises: (1) an analyte binding moiety that binds to the analyte; (2) an analyte binding moiety barcode that uniquely identifies an interaction between the analyte and the analyte binding moiety; and (3) an analyte capture sequence, wherein the analyte capture sequence binds to a capture domain; and (iii) a protein-binding molecule for immunofluorescence or immunohistochemistry; and (e) instructions for performing any of the methods disclosed herein.

In another aspect, this disclosure includes compositions that includes (a) a biological sample embedded in a polymer solution; (b) a non-aqueous droplet, wherein the non-aqueous droplet surrounds the biological sample; (c) one or more compositions to manipulate the biological sample, wherein the one or more compositions are selected from the group consisting of: (i) a plurality of capture probes, wherein a capture probe of the plurality comprises (i) a spatial barcode and (ii) a capture domain that binds to a sequence present in the analyte; (ii) a plurality of analyte capture agents, wherein an analyte capture agent of the plurality of analyte capture agents comprises: (1) an analyte binding moiety that binds to the analyte; (2) an analyte binding moiety barcode that uniquely identifies an interaction between the analyte and the analyte binding moiety; and (3) an analyte capture sequence, wherein the analyte capture sequence binds to a capture domain; and (iii) a protein-binding molecule for immunofluorescence or immunohistochemistry; and (d) a substrate comprising a plurality of probes that is capable of detecting the analyte, the capture probe, or the analyte capture agent.

In some embodiments, the compositions also include an analyte bound to the first and/or second capture probes. In some embodiments, the composition also includes an analyte bound to the first and/or second capture probes, where the capture probe has been extended using the captured analyte as a template (e.g., as a template in a nucleic acid extension reaction).

### EXAMPLES

### Example 1: Embedding of Sections of Biological Tissue and Manipulation of the Same

This example provides an exemplary method for partitioning a biological sample for manipulation and analysis. Referring to FIG. 9, a biological sample 901 is processed and sectioned into multiple tissue slides 902 using a cryostat, for example, but any sectioning method could be used. After generating multiple sections, the tissue sections are placed into a vial comprising a polymer solution 903. Upon placement into the polymer solution, the tissue sections are separated into distinct areas 904 in the vial. A non-aqueous oil solution 905 is added to the vial and the non-aqueous oil solution 905 surrounds the tissue sections, resulting in a number of polymer embedded tissue sections surrounded by oil in the vial.

A tissue section is then contacted with one or more compositions that can associate with an analyte in the biological sample. For instance, the tissue section is contacted with (1) a plurality of capture probes that hybridize to one or more mRNA molecules in the tissue section; (2) a plurality of analyte capture agents that associate with one or more proteins in a tissue section; or (3) a protein binding agent that can be identified using immunohistochemistry or immunofluorescence.

After manipulation of the tissue sections, the sections 907 are transferred to a substrate like a glass slide, a slide with wells, etc. 906, where the tissues if they are folded can unfold and be imaged using microscopy techniques 910. Alternatively, the tissues sections are placed in a capillary system 908 and imaged individually 909. After removing the tissue sections from the vial and imaging the tissue sections, the location and abundance of an analyte can be determined using analysis described herein 911.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of spatial analysis of a tissue sample, the method comprising:
(a) contacting the tissue sample with a substrate comprising a plurality of spatial capture probes,
wherein a spatial capture probe of the plurality of spatial capture probes comprises a spatial barcode and a spatial capture domain, wherein the spatial barcode identifies a location of the spatial capture probe on the substrate, wherein the spatial capture domain binds to an analyte in the tissue sample;
(b) releasing the plurality of spatial capture probes from the array such that the spatial capture probe associates with a location in the tissue sample;
(c) dissociating the tissue sample comprising the released spatial capture probes into a plurality of tissue subsamples; and
(d) partitioning the plurality of tissue subsamples into a plurality of partitions,
wherein a partition of the plurality of partitions comprises: a tissue subsample of the plurality of tissue subsamples and a subsample capture probe, wherein the subsample capture probe comprises a subsample barcode and a subsample capture domain, wherein the subsample barcode identifies the tissue subsample among the plurality of tissue subsamples, and wherein the subsample capture domain binds to the spatial capture domain.

2. The method of claim 1, wherein the spatial capture domain hybridizes to the analyte in the tissue sample.

3. The method of claim 1, further comprising permeabilizing the tissue sample to facilitate interaction between the spatial capture probe and the analyte.

4. The method of claim 1, wherein the partitioning the plurality of tissue subsamples in (d) comprises depositing the plurality of tissue subsamples in a polymer solution.

5. The method of claim 1, wherein the plurality of partitions is provided in a water-in-oil emulsion.

6. The method of claim 1, wherein the spatial capture domain hybridizes to the subsample capture domain.

7. The method of claim 6, further comprising extending the spatial capture probe using the subsample capture probe as a template.

8. The method of claim 6, further comprising extending the subsample capture probe using the spatial capture probe as a template.

9. The method of claim 1, wherein the partition further comprises an analyte capture probe comprising a sequence of the subsample barcode and an analyte capture domain, wherein the analyte capture domain hybridizes to the analyte or a proxy thereof.

10. The method of claim 9, further comprising extending the analyte capture probe using the analyte or the proxy thereof as a template.

11. The method of claim 9, further comprising lysing the plurality of partitions to facilitate hybridization of the analyte capture domain with the analyte or the proxy thereof.

12. The method of claim 1, further comprising performing nucleic acid amplification within the plurality of partitions.

13. The method of claim 1, further comprising determining and/or receiving the sequence of the spatial barcode or a complement thereof and the sequence of the subsample barcode or a complement thereof.

14. The method of claim 1, further comprising identifying the location of the analyte in the tissue sample based on the sequence of the spatial barcode or a complement thereof and the sequence of the subsample barcode or a complement thereof.

15. The method of claim 1, wherein the analyte is a nucleic acid.

16. The method of claim 1, wherein the analyte is a protein.

17. The method of claim 16, further comprising delivering a plurality of analyte capture agents to the tissue sample, wherein an analyte capture agent of the plurality of analyte capture agents comprises:
(i) an analyte binding moiety that binds to the protein;
(ii) an analyte binding moiety barcode that uniquely identifies an interaction between the protein and the analyte binding moiety; and
(iii) an analyte capture sequence, wherein the analyte capture sequence hybridizes to the capture domain.

18. The method of claim 1, wherein the spatial capture probe and/or the subsample capture probe is coupled to a bead.

19. The method of claim 1, wherein the tissue sample is a tissue section, and wherein the tissue subsample is a tissue subsection.

20. The method of claim 1, further comprising staining and/or imaging the tissue sample.
